(19) United States
(12) Patent Application Publication
(10) Pub. No.: US 2016/0362742 A1
(43) Pub. Date: Dec. 15, 2016

(54) METHOD AND SYSTEM FOR SEQUENCING IN CHARACTERIZATION OF ANTIBODY BINDING BEHAVIOR

(71) Applicant: uBiome, Inc., San Francisco, CA (US)

(72) Inventors: Zachary Apte, San Francisco, CA (US); Daniel Almonacid, San Francisco, CA (US); Jessica Richman, San Francisco, CA (US)

(21) Appl. No.: 15/183,652

(22) Filed: Jun. 15, 2016

Related U.S. Application Data
(60) Provisional application No. 62/175,748, filed on Jun. 15, 2015.

(51) Int. Cl. C12Q 1/68

(52) U.S. Cl. CPC C12Q 1/6874

(57) ABSTRACT

A method and system for characterization of antibody-bound targets by sequencing of synthetic oligonucleotides bound to the antibodies, the method including: receiving a sample having a set of antibodies; conjugating each of the set of antibodies with an oligonucleotide, thereby generating a set of oligonucleotide-conjugated antibodies; binding a first subset of the set of oligonucleotide-conjugated antibodies to a set of targets at a capture substrate; determining a sequence for at least one of: 1) each oligonucleotide of the first subset of oligonucleotide-conjugated antibodies that bind to the set of targets and 2) each oligonucleotide of a second subset of the set of oligonucleotide-conjugated antibodies that fail to bind to the set of targets; and generating an analysis of the sample from the sequences determined from at least one of the first and the second subsets of oligonucleotide-conjugated antibodies.

### CROSS-REFERENCE TO RELATED APPLICATIONS

This application claims the benefit of U.S. Provisional Application Ser. No. 62/175,748 filed 15 Jun. 2015, which is incorporated in its entirety herein by this reference.

### TECHNICAL FIELD

This invention relates generally to the field of molecular biology and more specifically to a new and useful method and system for nucleic acid sequencing in characterization of antibody binding behavior in the field of molecular biology.

### BACKGROUND

Characterization of antibody-binding behavior is useful in medical and research environments, with applications in health condition diagnostics, therapy design, and detection of toxic/harmful compounds in biological samples. In particular, improved characterization and detection methods could increase the efficiency and/or accuracy of diagnostic tests for disease panels, enhance diagnostics based upon non-nucleic acid biomarkers (e.g., blood biomarkers), and generally facilitate characterization of health states of biological samples. Current methods and systems for antibody sample handling, processing, characterization, and assaying in a high throughput manner are subject to deficiencies due to complications that arise when processing of samples in a multiplex and/or high throughput manner. Furthermore, current techniques for antibody binding characterization are expensive and are lacking in their ability to provide good limits of detection, due to inherent issues in current methods and systems for processing of biological samples.

As such, there is a need in the field of microbiology for a new and useful method and system for nucleic acid sequencing in characterization of antibody binding behavior. This invention creates such a new and useful method and system.

### BRIEF DESCRIPTION OF THE FIGURES

FIG. 1 is a flowchart of an embodiment of a method for characterization of antibody binding behavior;

FIG. 2 is a schematic of an embodiment of a method and system for characterization of antibody binding behavior;

FIG. 3 depicts an example of a lateral flow assay capture substrate in an embodiment of a method for characterization of antibody binding behavior;

FIGS. 4 and 5 depict variations of portions of an embodiment of a method for characterization of antibody binding behavior;

FIG. 6 is a schematic of an embodiment of a system for characterization of antibody binding behavior; and

FIGS. 7A and 7B are schematics of portions of embodiments of a system for characterization of antibody binding behavior.

### DESCRIPTION OF THE EMBODIMENTS

The following description of the embodiments of the invention is not intended to limit the invention to these embodiments, but rather to enable any person skilled in the art to make and use this invention.

### 1. Method

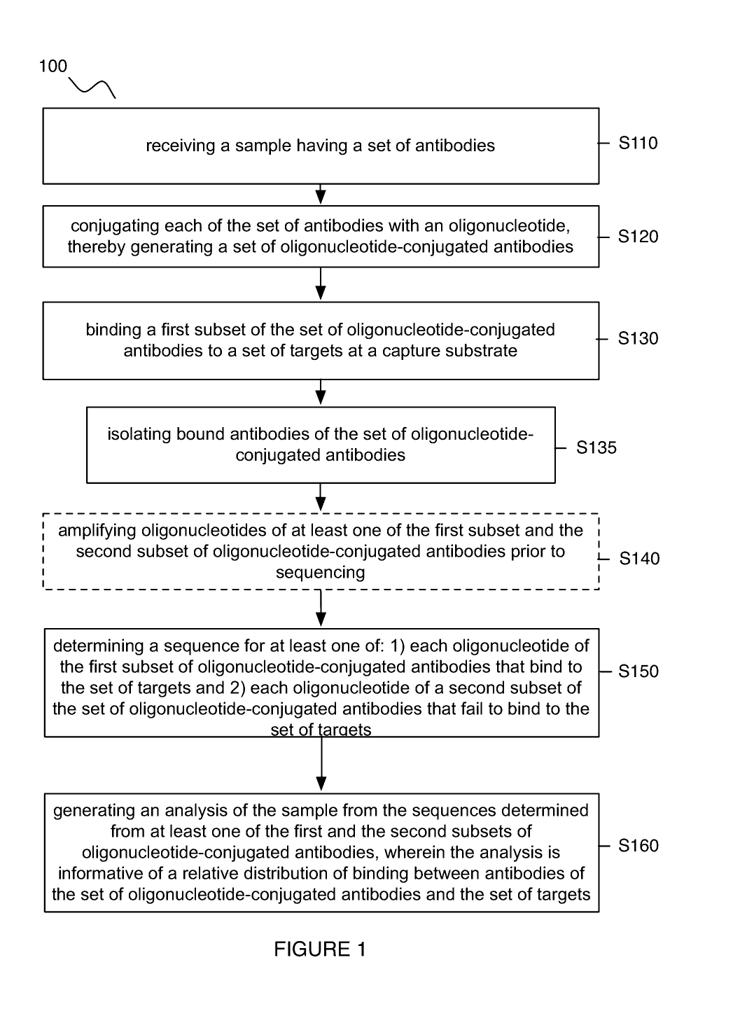

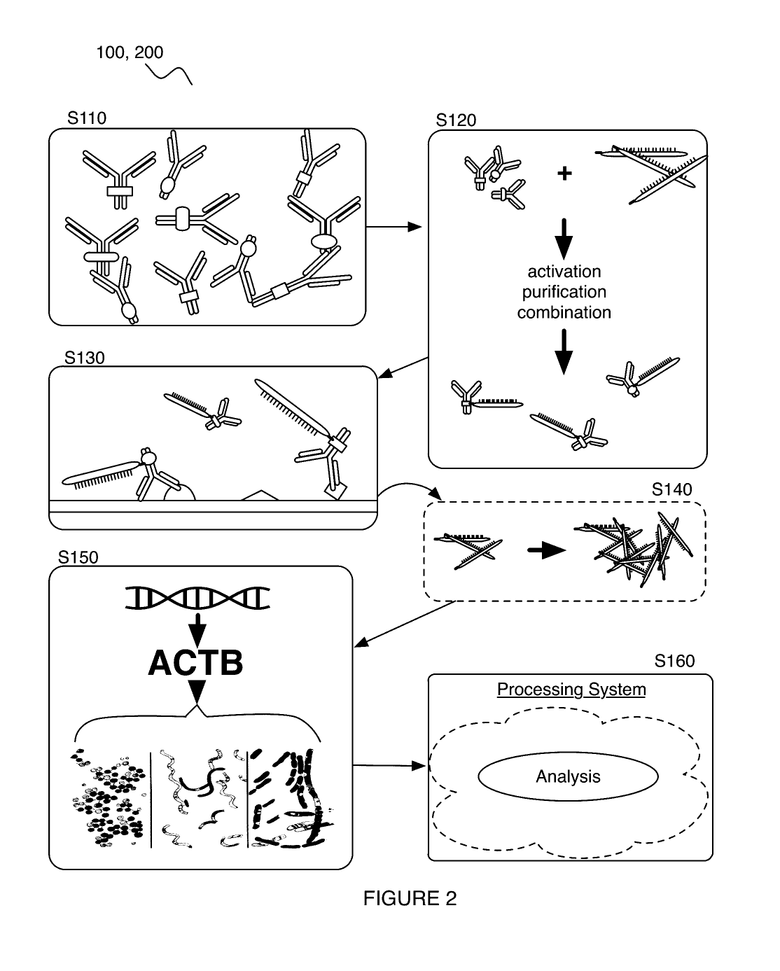

As shown in FIGS. 1 and 2, an embodiment of a method 100 for characterization of antibody binding behavior includes: receiving a sample having a set of antibodies S110; conjugating each of the set of antibodies with an oligonucleotide, thereby generating a set of oligonucleotide-conjugated antibodies S120; binding a first subset of the set of oligonucleotide-conjugated antibodies to a set of targets at a capture substrate S130; determining a sequence for at least one of: 1) each oligonucleotide of the first subset of oligonucleotide-conjugated antibodies that bind to the set of targets and 2) each oligonucleotide of a second subset of the set of oligonucleotide-conjugated antibodies that fail to bind to the set of targets S150; and generating an analysis of the sample from the sequences determined from at least one of the first and the second subsets of oligonucleotide-conjugated antibodies, wherein the analysis is informative of a relative distribution of binding between antibodies of the set of oligonucleotide-conjugated antibodies and the set of targets. In some variations, the method 100 can also include: amplifying oligonucleotides of at least one of the first subset and the second subset of oligonucleotide-conjugated antibodies prior to sequencing S140.

The method 100 functions to facilitate characterization of a combination of nucleic acid segments in a sample, in parallel, and in a rapid manner with improved limits of detection. In particular, the method 100 can function to enable determination of any molecule bound by an antibody, using sequencing (or other polymerase chain reaction-based techniques) of an oligonucleotide associated with the antibody. In a multiplexed manner, the method 100 can function to enable determination of any set of molecules bound by a set of antibodies, using sequencing (or other polymerase chain reaction-based techniques) of a set of oligonucleotides associated with the set of antibodies. In a specific application associated with 16S V4 targets (e.g., in relation to microbiome sequencing), the oligonucleotides of the method 100 can implement synthetic (i.e., “alien”) DNA that includes a region recognized by 16S V4 primers, wherein the remainder of the synthetic DNA is different (i.e., non-existent in nature). As such, in using primers designed for the 16S region(s), the method 100 can enable simultaneous amplification of all bacterial and archaeal DNA present in a sample, in addition to the “alien” 16S-like oligonucleotides that are linked to antibodies that are bound to an antigen/target. Thus, identification of DNA/RNA, proteins, small molecules, and/or any other suitable molecules is reduced to a DNA sequencing process according to the method 100. Variations of the “alien” oligonucleotides and/or primers can be adapted to any other suitable region (e.g., 18S region, ITS region, etc.) in relation to amplification, processing, and/or identification of any other suitable target(s) in a sample.

As such, in using antibodies tagged with nucleic acids (e.g., oligonucleotides) the method 100 can be used to detect, identify, and characterize binding between antibody and antigens/targets with a DNA sequencing process, wherein the targets can be molecules associated with one or more of: infectious agents (e.g., prions), microorganisms (e.g., bacteria, viruses, fungal organisms, etc.), toxic compounds, and any other suitable antibody target associated with medical (e.g., diagnostic, therapeutic) or research applications. In one application, the method 100 can be used to detect which of a set of antibody-bound oligonucleotides in a sample bind to targets (e.g., in an enzyme-linked immunosorbent assay), with characterization of a relative distribution of binding. Variations of the method 100 can additionally or alternatively be used to support diagnostic tests for disease panels (e.g., respiratory disease panels, sexually-transmitted disease panels, etc.) and any other suitable biomarker-based test, where detection and quantification (i.e., relative quantification, absolute quantification) of a biocompound is desired.

In one variation, the method 100 can be used to process and generate analyses that provide information regarding which of a set of diverse antibodies bind to the target(s)/antigen(s) of an immunoassay. Additionally or alternatively, the method 100 can provide a rapid and novel diagnostic tool for detection of multiple antigen targets in parallel, in a multiplex manner, and in an efficient manner. As such, as indicated above, the output(s) of the method 100 can provide diagnostic information associated with one or more: infectious agents (e.g., prions), microorganisms (e.g., bacteria, viruses, fungal organisms, etc.), toxic compounds, and any other suitable antibody target associated with medical (e.g., diagnostic, therapeutic) or research applications using a multiplexed assay. In examples, the output(s) of the method 100 can support diagnostic tests for disease panels (e.g., respiratory disease panels, sexually-transmitted disease panels, etc.) and any other suitable disease panel using a diverse set of oligonucleotide-tagged antibodies.

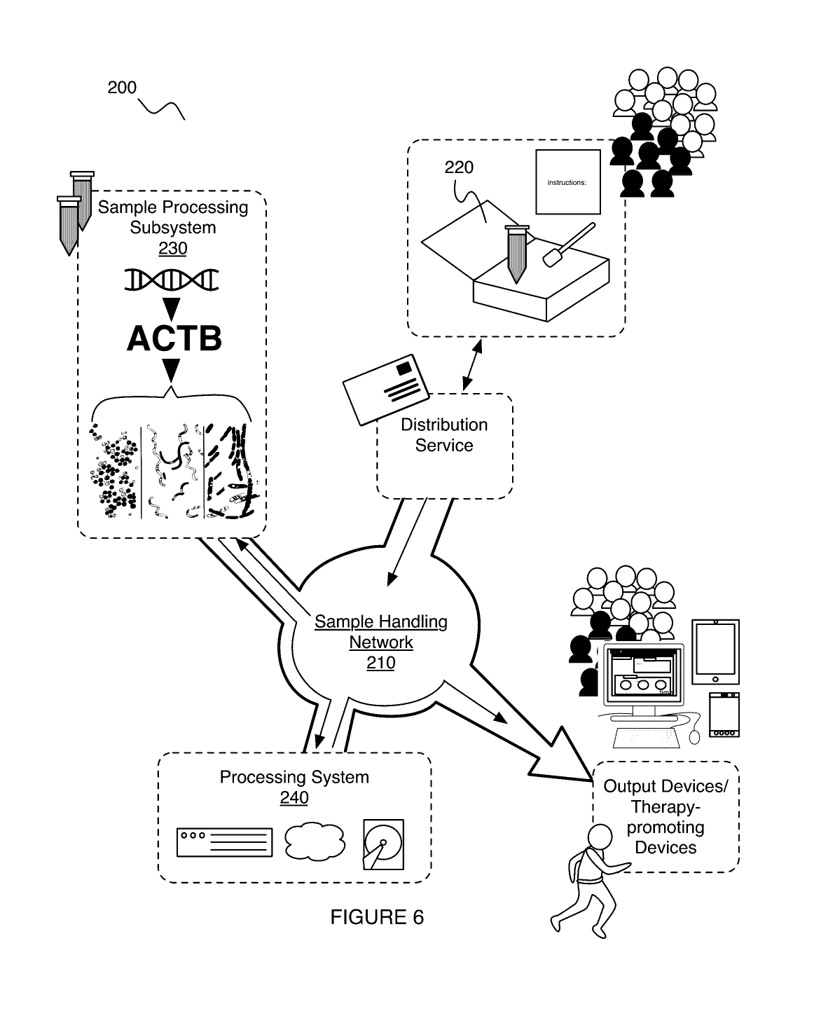

In applications, the method 100 can be implemented, at least in part, at a system 200 (as shown in FIGS. 2 and 6) that receives a biological sample having a distribution of different antibody-bound oligonucleotides, binds at least a portion of the antibody-bound oligonucleotides to targets at a substrate, amplifies the oligonucleotides of the antibody-bound oligonucleotides bound to the targets, and sequences the oligonucleotides of the antibody-bound oligonucleotides bound to the targets. The system 200 can then be configured to generate an analysis of binding behavior between specific antibody-bound oligonucleotides and the targets at the substrate, which is indicative of relative distributions of binding associated with the different antibody-bound oligonucleotides of the biological sample. The method 100 can, however, alternatively be implemented using any other suitable system(s) configured to receive and process biological samples having nucleic acid components, where sequencing of the nucleic acid components is of interest.

### 1.1 Method—Antibody Sample Reception and Processing

Block S110 recites: receiving a sample having a set of antibodies, which functions to provide a set of candidate antibodies for binding to at least one target/antigen at a substrate in Block S130. In particular, the set of antibodies is preferably a diverse set of antibodies that have selective binding affinity to antigens/target compounds in Block S130, such that a portion of the set of antibodies binds to the antigen(s) in Block S130, while another portion of the set of antibodies fails to bind to the antigen(s). The sample preferably comprises the set of antibodies in solution, wherein each of the set of antibodies includes an antigen binding site and a conjugation site for conjugation to an oligonucleotide. The set of antibodies can include one or more of: monoclonal antibodies, polyclonal antibodies, naturally produced antibodies, and genetically engineered antibodies. Furthermore, the set of antibodies can include antibodies sourced from any suitable species. Furthermore, the set of antibodies can be distributed across any of five different isotypes (i.e., IgA, IgD, IgE, EgG, IgM) based upon their Fc regions. Furthermore, the set of antibodies can include antibodies having similar hypervariable regions or dissimilar hypervariable regions. Furthermore, one or more of the set of antibodies can exhibit a degree of class switching behavior, in forming a different isotype of the antibody while retaining an antigen-specific variable region for selective binding to an antigen of interest in Block S130. Furthermore, the set of antibodies can include whole antibodies and/or fragmented antibodies (e.g., antibodies that have undergone partial enzymatic digestion). As such, the set of antibodies of the biological sample of Block S110 can include any suitable type of antibody. Additionally or alternatively, variations of the method 100 can be adapted to molecule pairs that bind or otherwise couple with specificity (e.g., high specificity). As such, the set of antibodies of Block S110 and the targets/antigens of Block S130 can be substituted with other molecules (e.g., synthetic “antibodies”, synthetic antigens) that couple with specificity in other variations of the method 100.

The set of antibodies of Block S110 can thus comprise different types of antibodies configured for binding with different targets, wherein there is a distribution of each of the different types of antibodies in the sample of Block S110. Additionally or alternatively, the set of antibodies in Block S110 can include a single type of antibody, such that there is no relative abundance between different antibody types in the sample.

In variations, Block S110 can include steps for preprocessing the sample in a manner that prevents antibodies of the set of antibodies from interacting with each other (e.g., coupling with each other) in an undesired manner. For instance, Block S110 can include preprocessing the sample including the set of antibodies with blocking compounds that controllably block antibody sites that have potential for interacting with other antibodies of the set of antibodies in an undesired manner. Additionally or alternatively, Block S110 can include preprocessing the sample including the set of antibodies with one or more solvents that reduce interactions between antibodies in an undesired manner. Additionally or alternatively, Block S110 can include providing sufficient sample volume (e.g., solution volume) to control a concentration of antibodies within the sample, thereby limiting interactions between antibodies of the set of antibodies in the sample. Additionally or alternatively, Block S110 can comprise selectively omitting antibodies from the sample or selectively including antibodies in the sample based upon interaction behavior (e.g., including antibodies that reduce interactions between different antibodies of the set of antibodies), thereby selectively designing the antibody distribution profile of the set of antibodies of the sample. Similarly, Block S110 can include selectively omitting antibody fragments from the sample or selectively including antibody fragments in the sample based upon interaction behavior. Additionally or alternatively, Block S110 can include application of antibodies (or antibody subsets) of the set of antibodies in stages (e.g., in sequence). For instance, a first antibody group could be applied and allowed to bind to an antigen, followed by a wash step to remove unbound instances of the first antibody group. Then, a second antibody group could be applied and allowed to bind to an antigen, followed by a wash step to remove unbound instances of the second antibody group. This process could then be repeated for subsequent antibody groups (e.g., in a microfluidic device, etc.). However, prevention of undesired antibody-antibody interactions can be implemented in Block S110 in any other suitable manner.

While the above steps are described prior to steps related to conjugation of each of the set of antibodies with an oligonucleotide, preventing antibodies of the set of antibodies from interacting with each other (e.g., coupling with each other) in an undesired manner with buffers or any other suitable processing elements can additionally or alternatively be performed at any other suitable stage of the method 100. For instance, processing steps associated with a capture substrate (e.g., capture plate, capture antibody) of Block S130 can be additionally or alternatively be implemented to prevent undesired antibody-antibody interactions.

Furthermore, variations of Block S110 can additionally or alternatively include steps that enhance purification of the sample of antibodies. For instance, Block S110 can include one or more of: desalting the sample, centrifuging the sample, performing filtration of the sample, performing a buffer exchange step with the sample, performing chromatographic separation on the sample (e.g., with affinity chromatography), and performing any other purification step to control purity of antibodies of the set of antibodies of the sample in a characterizable manner. Additionally or alternatively, variations of Block S110 can include steps that activate conjugation and/or binding sites of the set of antibodies to facilitate downstream portions of the method 100 related to antibody-oligonucleotide conjugation and/or antibody-antigen binding.

Block S110 is preferably implemented using a system comprising apparatus, control subsystems, and actuators that enable reception of samples, wet lab processing of samples, data extraction from samples, and/or data processing for samples with reduced human input (e.g., in an automated manner). In some variations, such systems can include one or more of: sample container handling robotics (e.g., gantries, robotic arm systems having desired degrees of freedom and dexterity, control system), fluid delivery apparatus (e.g., aspiration systems, fluid conduits, control systems), sample analysis apparatus (e.g., optical detection systems, signal transmission units), computing systems (e.g., data storage units, remote servers for data processing, benchtop data processing computers, cloud-based computing systems, etc.), and any other suitable apparatus for semi-automated or automated sample handling. However, Block S110 can additionally or alternatively be implemented entirely by a human or other entity.

1.2 Method—Antibody Conjugation with Oligonucleotides

Block S120 recites: conjugating each of the set of antibodies with an oligonucleotide, thereby generating a set of oligonucleotide-conjugated antibodies. Block S120 functions to generate a set of oligonucleotide-conjugated antibodies that can be processed in parallel to determine characteristic sequences of the oligonucleotides and enable identification of selective binding behavior of the antibodies bound to the oligonucleotides, to antigens/targets in Block S130. As such, the oligonucleotides used in Block S120 can enable identification of any antibody-bound targets upon sequencing of the oligonucleotides, in a singular or in a multiplex manner (e.g., using a set of oligonucleotides).

In one application associated with 16S V4 targets (e.g., in relation to microbiome sequencing), the oligonucleotides of Block S120 can comprise synthetic (i.e., “alien”) DNA that includes a region recognized by 16S V4 primers, wherein the remainder of the synthetic DNA is different (i.e., non-existent in nature). As such, in using primers designed for the 16S region(s), subsequent blocks of the method 100 can enable simultaneous amplification of all bacterial and archaeal DNA present in a sample, in addition to the “alien” 16S-like oligonucleotides that are linked to antibodies, wherein the antibodies are bound or become bound to an antigen/target molecule. Thus, identification of DNA/RNA, proteins, small molecules, and/or any other suitable molecules can be reduced to a DNA sequencing process based on the oligonucleotides used in Block S120. Variations of the “alien” oligonucleotides and/or primers associated with Block S120 can, however, be adapted to any other suitable region (e.g., 18S region, ITS region, etc.) in relation to amplification, processing, and/or identification of any other suitable target(s) in a sample.

In Block S120, the oligonucleotides are preferably covalently bonded to associated antibodies of the set of antibodies of Block S110; however, the oligonucleotides can be bonded or attached to the associated antibodies of the set of antibodies of Block S110 in any other suitable manner, in a reversible or an irreversible manner. In Block S120, conjugation can comprise one or more of: mixing the set of antibodies with oligonucleotides in solution, incubating the set of antibodies with oligonucleotides in solution, heating the set of antibodies with oligonucleotides in solution, agitating (e.g., rocking, shaking) the set of antibodies with oligonucleotides in solution, washing the set of antibodies with oligonucleotides in solution and performing any other suitable technique for coupling antibodies to oligonucleotides. Furthermore, in Block S120, conjugation is preferably conducted unidirectionally in order to form antibody-oligonucleotide complexes without formation of antibody-antibody complexes and/or oligonucleotide-oligonucleotide complexes. Furthermore, conjugation can be performed with controls (e.g., positive controls, negative controls, etc.) to enable determination that conjugation is occurring properly. However, conjugation can be performed in Block S120 in any other suitable manner.

In Block S120, the oligonucleotides preferably have lengths of from 20-120 bases; however, the oligonucleotides can alternatively have any other suitable length (e.g., up to 200 bases). Furthermore an oligonucleotide of the oligonucleotides used in Block S120 is preferably configured to couple to an associated antibody of the set of antibodies at the 5′ end of the oligonucleotide, which provides an increase in conjugation efficiency; however, an oligonucleotide used in Block S120 can additionally or alternatively be configured to couple to an associated antibody of the set of antibodies at the 3′ or the 5′ end of the oligonucleotide. The oligonucleotides of Block S120 preferably also include a terminal amino group (e.g., an amino group attached to the oligonucleotide by a hydrocarbon chain); but can additionally or alternatively be configured with any other suitable functional group(s). For instance, modifications to an oligonucleotide of Block S120 can include one or more of: phosphorylation modifications (e.g., for using the oligonucleotide as a DNA ligase substrate), linker modifications (e.g., biotins, amino-modifiers, azides, cholesteryl-TEG, Alkynes, Thiols, etc. to enhance attachment to a substrate), fluorophore modifications, dark quencher modifications, blocking modifications (e.g., to prevent steric hindrance effects), base modifications (e.g., to modulate hybridization affinity), phosphorothioate bond modifications (e.g., to prevent nuclease degradation), aldehyde modifications, hydrazine modifications, and any other suitable modification.

Similar to Block S110, variations of Block S120 can additionally or alternatively include steps that enhance purification of the oligonucleotides used. For instance, Block S120 can include one or more of: chromatographic processing of oligonucleotides (e.g., with high performance liquid chromatography), desalting the sample of oligonucleotides, washing the sample of oligonucleotides, centrifuging the sample of oligonucleotides, and performing any other purification step to control purity of oligonucleotides in a characterizable manner. Additionally or alternatively, variations of Block S110 can include steps that activate conjugation sites of the oligonucleotides to facilitate antibody-oligonucleotide conjugation.

Furthermore, to enhance conjugation efficiency and/or to prevent undesired issues during conjugation, Block S120 can include one or more of: eliminating compounds with functional groups (e.g., Thiols) that compete for conjugation sites, reducing or minimizing base repetition (e.g., as in homopolymers) in the oligonucleotide(s), using or designing oligonucleotides with ≦4 sequential G bases, which can form a structure (e.g., quadruplex, cruciform, etc.) that reduces hybridization and coupling efficiencies, and any other step that resolves conjugation errors. Furthermore, Block S120 can include steps that remove unbound or fragmented oligonucleotides (e.g., by precipitation and reconstitution of a conjugate) or otherwise post-processes the sample of oligonucleotide-conjugated antibodies in any other suitable manner.

In Block S120, and in relation to Block S140, each of the oligonucleotide barcodes used can include one or more elements that facilitate sequencing in Block S150. For instance, in relation to Illumina sequencing, each oligonucleotide of Block S120 can include one or more of: a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, a sequence for targeting a specific target region (e.g., a target in an oligonucleotide attached to an antibody, a predesigned target in all oligonucleotides coupled to the set of antibodies, etc.), a reverse index sequence (e.g., corresponding to an Illumina reverse index for MiSeq/NextSeq/HiSeq platforms), and a reverse barcode sequence. Thus, variations of the method 100 can include sequencing directly after binding and identification of bound antibodies in Block S130, without amplification in optional Block S140. In another variation associated with Blocks S120 and S140, Block S140 can be used after Block S120 to add one or more elements to oligonucleotide barcodes for sequencing, wherein the oligonucleotides used in Block S120 already include a Nextera sequence component. Variations of Blocks S120 and S140 can, however, be used in any other suitable manner, including implementations in any other suitable sequencing technology.

Similar to Block S110, Block S120 can be implemented using a system comprising apparatus, control subsystems, and actuators that enable reception of samples, wet lab processing of samples, data extraction from samples, and/or data processing for samples with reduced human input (e.g., in an automated manner), in relation to conjugation of antibodies with oligonucleotides. In some variations, such systems can include one or more of: sample container handling robotics (e.g., gantries, robotic arm systems having desired degrees of freedom and dexterity, control system), fluid delivery apparatus (e.g., aspiration systems, fluid conduits, control systems), sample analysis apparatus (e.g., optical detection systems, signal transmission units), computing systems (e.g., data storage units, remote servers for data processing, benchtop data processing computers, cloud-based computing systems, etc.), and any other suitable apparatus for semi-automated or automated sample handling. However, Block S120 can additionally or alternatively be implemented entirely by a human or other entity.

### 1.3 Method—Antibody-Target Binding

Block S130 recites: binding a first subset of the set of oligonucleotide-conjugated antibodies to a set of targets at a capture substrate, which functions to selectively bind a subset of the set of oligonucleotide-conjugated antibodies to antigen targets and/or directly to a capture substrate. Preferably, binding in Block S130 occurs contemporaneously (e.g., simultaneously, at approximately the same time) between the set of oligonucleotide-conjugated antibodies to a set of targets; however, binding can alternatively not occur contemporaneously in Block S130. In binding the first subset of the set of oligonucleotide-conjugated antibodies to a set of targets (e.g., antigens), Block S130 is preferably performed in an immunoassay. In variations, the immunoassay can have any substrate format, can be performed in any suitable number of stages (e.g., with intermediate washing steps), and/or can be heterogeneous or homogenous. In one variation, Block S130 can be implemented with an enzyme-linked immunosorbent assay (ELISA), whereby the target(s)/antigen(s) are immobilized on a solid support (e.g., plate, microtiter plate, etc.) non-specifically or specifically (e.g., with a capture antibody as in a sandwich ELISA). In this variation, the ELISA can be run in a qualitative or a quantitative format. Furthermore, the ELISA can be performed with any suitable number of binding stages (e.g., to bind subsets of the set of oligonucleotide-conjugated antibodies in stages in order to limit undesired antibody-antibody interactions), with any suitable number of intermediate washing or processing steps. As such, the washing steps can remove unbound antibodies, thereby enabling identification of the antibodies that bind to targets (and complementarily, antibodies that fail to bind to targets). Additionally or alternatively, the ELISA can be a competitive ELISA or any other suitable type of ELISA (e.g., MELISA).

In another variation, Block S130 can alternatively be implemented with a lateral flow assay (e.g., competitive lateral flow assay, sandwich lateral flow assay), whereby a sample pad (e.g., porous substrate) receives a sample and transmits it toward a conjugate pad, by capillary action, for binding of antigen(s)/target(s) to the set of oligonucleotide-conjugated antibodies. In this variation, oligonucleotide-conjugated antibodies that bind to antigen(s)/target(s) post performance of the assay can be detected and sequenced in Block S140. The conjugate pad used in the lateral flow assay can have test regions (e.g., a test strip oriented perpendicular to a flow direction through the conjugate pad), and can additionally include a control region (e.g., control strip) that facilitates detection of proper performance of the assay. In this variation, the lateral flow assay can be performed with any suitable number of binding stages (e.g., to bind subsets of the set of oligonucleotide-conjugated antibodies in stages in order to limit undesired binding interactions), with any suitable number of intermediate washing or processing steps. Alternatively, in other variations, the immunoassay implemented in Block S130 can comprise one or more of: a cloned enzyme donor immunoassay (CEDIA), a magnetic immunoassay, a radioimmunoassay, a surround optical fiber immunoassay (SOFIA), and any other suitable assay.

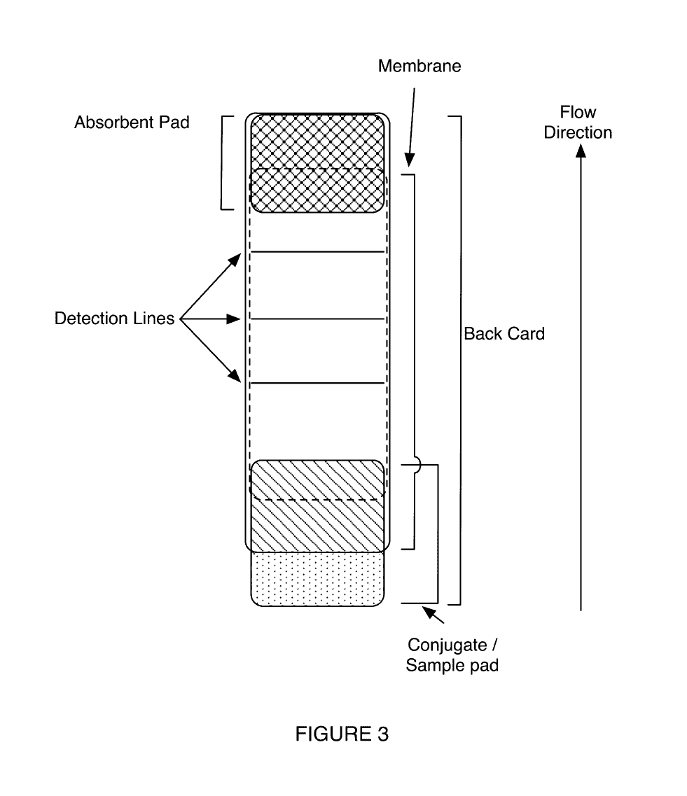

In a specific example of a capture substrate for a lateral flow assay, as shown in FIG. 3 the capture substrate can include: a membrane including a detection region, an absorbent region coupled to a downstream end of the membrane (in order to facilitate flow through the membrane), a sample pad disposed at an upstream end of the membrane and configured to receive and transmit the test sample through a conjugate pad and into the membrane, and a back card that serves as a substrate for the membrane, wherein the back card can couple to a cover that functions to maintain positions of elements of the lateral flow element. The sample pad can thus wick a sample (e.g., a sample including the set of targets) to a conjugate pad for antibody-target binding in subsequent blocks of the method 100.

In any of the above assays of Block S130, surface features of a substrate (e.g., plate, microtiter plate, lateral flow substrate, etc.) can be designed to prevent undesired interactions between antibodies and/or the substrate. For instance, surface chemistry, charge, roughness, porosity, and any other suitable parameter can be modulated to control specificity of sticking between antibodies and the substrate. However, Block S130 can include any other suitable blocks or steps that prevent or reduce undesired interactions (e.g., biases in binding preference) between antibodies of the set of oligonucleotide-conjugated antibodies and the substrate.

In relation to the set of targets, Block S130 can include Block S131, which recites: receiving a test sample from a subject (e.g., a human subject, a non-human subject) at an appropriate capture substrate, wherein the test sample includes the set of targets. Block S131 can include any suitable sample reception and/or processing steps. For instance, variations of non-invasive manners of sample reception can use any one or more of: a permeable substrate (e.g., a swab configured to wipe a region of a subject's body, toilet paper, a sponge, etc.), a non-permeable substrate (e.g., a slide, tape, etc.), a container (e.g., vial, tube, bag, etc.) configured to receive a sample from a region of a subject's body, and any other suitable sample-reception element. In a specific example, samples can be collected from one or more of a subject's nose, skin, genitals, mouth, and gut in a non-invasive manner (e.g., using a swab and a vial). However, a sample of the subject can additionally or alternatively be received in Block S131 in a semi-invasive manner or an invasive manner. In variations, invasive manners of sample reception can use any one or more of: a needle, a syringe, a biopsy element, a lance, and any other suitable instrument for collection of a sample in a semi-invasive or invasive manner. In specific examples, samples can comprise blood samples, plasma/serum samples (e.g., to enable extraction of cell-free DNA), cerebrospinal fluid, and tissue samples.

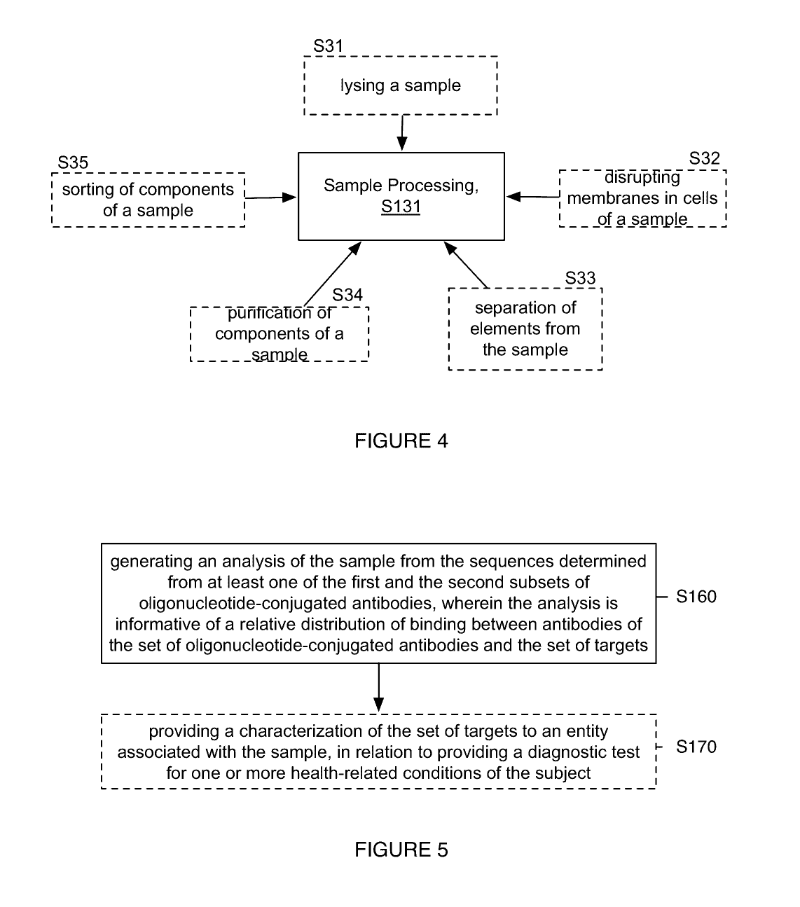

In variations, as shown in FIG. 4, sample processing in Block S131 can thus include any one or more of: lysing a sample S31, disrupting membranes in cells of a sample S32, separation of elements (e.g., RNA, proteins) from the sample S33 in order to expose the set of targets, purification of desired components of the sample (e.g., nucleic acids, antigens, other proteins, etc.) in a sample S34, sorting of components of the sample S35, and any other suitable processing steps.

In variations, lysing a sample S31 and/or disrupting membranes in cells of a sample S32 preferably includes physical methods (e.g., bead beating, nitrogen decompression, homogenization, sonication) of cell lysing/membrane disruption, which omit certain reagents that produce bias in representation of certain components upon sequencing. Additionally or alternatively, lysing or disrupting in Blocks S31 or S32 can involve chemical methods (e.g., using a detergent, using a solvent, using a surfactant, etc.). Blocks S31 and S32 can thus function to complete lysis of components of a sample, in variations wherein the sample has been received at the sample handling network in a pre-processed state of lysis. In variations, separation of elements from the sample S33 can include removal of RNA using RNases and/or separations of proteins using proteases. In variations, purification of nucleic acids in a sample to generate a nucleic acid sample S34 can include one or more of: precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and any other suitable purification techniques. However, any suitable processing steps can be performed in relation to Block S131.

After Block S130 is performed, detection and characterization of different antibody-antigen complexes in parallel is preferably performed during downstream sequencing of the subset of oligonucleotides coupled to the antigen-bound antibodies and/or subset of oligonucleotides coupled to antibodies that fail to bind to antigens (i.e., in Block S150); however, detection and characterization of antibody-antigen complexes can additionally or alternatively be validated or normalized based upon coupling of a probe (e.g., fluorescent probe, color-changing probe, color-exhibiting probe, etc.) to the antibody-antigen complexes formed in the immunoassay(s) of Block S130. Additionally or alternatively, detection and characterization of antibody-antigen complexes can be performed in relation to Blocks S130 and S150 in any other suitable manner.

Furthermore, Block S130 can additionally or alternatively include Block S135, which recites: isolating bound antibodies of the set of oligonucleotide-conjugated antibodies prior to subsequent blocks of the method 100, which functions allow isolation in relation to method steps of one or more of: amplification, sequencing, and analysis. Isolation in Block S135 can comprise any suitable type of: washing process, rinsing process, elution process, sorting process, and/or any other suitable process associated with isolation of bound antibodies.

Similar to previously described blocks, Block S130 can be implemented using a system comprising apparatus, control subsystems, and actuators that enable reception of samples, wet lab processing of samples, data extraction from samples, and/or data processing for samples with reduced human input (e.g., in an automated manner), in relation to binding antibodies to targets at capture substrates. In some variations, such systems can include one or more of: sample container handling robotics (e.g., gantries, robotic arm systems having desired degrees of freedom and dexterity, control system), substrate (e.g., immunoassay substrate) handling robotics, fluid delivery apparatus (e.g., aspiration systems, fluid conduits, control systems), sample analysis apparatus (e.g., optical detection systems, signal transmission units), computing systems (e.g., data storage units, remote servers for data processing, benchtop data processing computers, cloud-based computing systems, etc.), and any other suitable apparatus for semi-automated or automated sample handling. However, Block S130 can additionally or alternatively be implemented entirely by a human or other entity.

### 1.4 Method—Oligonucleotide Amplification

As indicated above, some variations of the method 100 can additionally or alternatively include Block S140, which recites: amplifying oligonucleotides of at least one of the first subset of oligonucleotide-conjugated antibodies and a second subset of oligonucleotide-conjugated antibodies (e.g., oligonucleotide-conjugated antibodies that fail to bind to the set of targets) prior to sequencing. Block S140 functions to amplify oligonucleotides post processing in Block S130, which can facilitate signal enhancement and/or provide good limits of detection upon sequencing oligonucleotides in Block S150. Block S140 can additionally or alternatively function to amplify the oligonucleotide tags with primers that append sequencing elements to the oligonucleotides in a manner that facilitates sequencing in Block S150. In particular, in relation to Illumina sequencing, amplification in Block S140 can involve primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, a sequence for targeting a specific target region (e.g., a target in an oligonucleotide attached to an antibody, a predesigned target in all oligonucleotides coupled to the set of antibodies, etc.), a reverse index sequence (e.g., corresponding to an Illumina reverse index for MiSeq/NextSeq/HiSeq platforms), and, optionally, a reverse barcode sequence. However, the primers used for amplification can additionally or alternatively have any other suitable functional elements that facilitate downstream processing and analysis according to the method 100.

In particular, Blocks S140 and S150 preferably include amplification of oligonucleotides of the first subset of oligonucleotide-conjugated antibodies that bind to targets in Block S130, which can be used to characterize the oligonucleotide-conjugated antibodies that bind in a direct manner. However, Blocks S140 and S150 can additionally or alternatively include amplification of oligonucleotides of a second subset of oligonucleotide-conjugated antibodies that fail to bind in Block S130, which can be used to characterize the oligonucleotide-conjugated antibodies that bind in an indirect manner. As such, characterization of binding for the set of oligonucleotide-conjugated antibodies can be performed in a direct and/or in an indirect manner.

In variations of Block S140, amplification of oligonucleotides preferably includes one or more of: polymerase chain reaction (PCR)-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification technique. In amplification of purified nucleic acids, the primers used are preferably designed to universally amplify all of the oligonucleotide barcodes that are coupled to the antibodies, thereby enabling amplification of the barcodes for sequencing, and enabling identification of the antibodies that bound to antigens/targets in Block S130. Additionally or alternatively, the primers can be selected to prevent or minimize amplification bias, as well as configured to amplify nucleic acid regions/sequences (e.g., of the 16S region, the 18S region, the ITS region, etc.) that are informative taxonomically, phylogenetically, for diagnostics, for formulations (e.g., for probiotic formulations), and/or for any other suitable purpose. Thus, universal primers configured to avoid amplification bias can be used in amplification. Primers used in variations of Block S140 can additionally or alternatively include incorporated barcode sequences specific to each sample, which can facilitate identification of biological samples post-amplification. As indicated above, primers used in variations of Block S140 can additionally or alternatively include adaptor regions configured to cooperate with sequencing techniques involving complementary adaptors (e.g., Illumina Sequencing). Additionally or alternatively, Block S140 can implement any other step configured to facilitate processing (e.g., using a Nextera kit for fragmentation, etc.).

Similar to previously described blocks, Block S140 can be implemented using a system comprising apparatus, control subsystems, and actuators that enable reception of samples, wet lab processing of samples, data extraction from samples, and/or data processing for samples with reduced human input (e.g., in an automated manner), in relation to amplification. In some variations, such systems can include one or more of: sample container handling robotics (e.g., gantries, robotic arm systems having desired degrees of freedom and dexterity, control system), fluid delivery apparatus (e.g., aspiration systems, fluid conduits, control systems), thermocycling apparatus (e.g., heaters and heater control modules), sample analysis apparatus (e.g., optical detection systems, signal transmission units), computing systems (e.g., data storage units, remote servers for data processing, benchtop data processing computers, cloud-based computing systems, etc.), and any other suitable apparatus for semi-automated or automated sample handling. However, Block S140 can additionally or alternatively be implemented entirely by a human or other entity.

### 1.5 Method—Sequencing

Block S150 recites: determining a sequence for at least one of: 1) each oligonucleotide of the first subset of oligonucleotide-conjugated antibodies that bind to the set of targets and 2) each oligonucleotide of a second subset of the set of oligonucleotide-conjugated antibodies that fail to bind to the set of targets. Block S150 functions to sequence the oligonucleotide tags of the antibody-oligonucleotide complexes in parallel, thereby enabling characterization of which antibody-oligonucleotide complexes bind or fail to bind to target antigens of the immunoassay of Block S130. In sequencing, Block S150 thus allows the method 100 to enable identifying of any molecule (e.g., target, antigen) bound to an antibody, using a sequencing process. Block S150 preferably includes sequencing of oligonucleotides of the first subset of oligonucleotide-conjugated antibodies that bind to targets in Block S130, which can be used to characterize the oligonucleotide-conjugated antibodies that bind in a direct manner. However, as indicated above, if aspects of the entire set of oligonucleotide-conjugated antibodies are known, Block S150 can additionally or alternatively include sequencing of oligonucleotides of a second subset of oligonucleotide-conjugated antibodies that fail to bind in Block S130, which can be used to characterize the oligonucleotide-conjugated antibodies that bind in an indirect manner. As such, characterization of binding for the set of oligonucleotide-conjugated antibodies can be performed in a direct and/or in an indirect manner. Furthermore, variations of the method 100 can alternatively include the sequencing step of Block S150 without implementation of the amplification step of Block S140.

In Block S150, sequencing can further be performed on any suitable molecule (e.g., chain, fragment, etc.), as indicated throughout the disclosure. In the specific example, sequencing comprises Illumina sequencing (e.g., with a HiSeq platform, with a MiSeq platform, with a NextSeq platform, etc.) using a sequencing-by-synthesis technique. Additionally or alternatively, any other suitable next generation sequencing technology (e.g., PacBio platform, MinION platform, Oxford Nanopore platform, etc.) can be used. Additionally or alternatively, any other suitable sequencing platform or method can be used (e.g., a Roche 454 Life Sciences platform, a Life Technologies SOLiD platform, etc.). In examples, sequencing can include deep sequencing to quantify the number of copies of a particular sequence in a sample and then also be used to determine the relative abundance of different sequences in a sample. Deep sequencing refers to highly redundant sequencing of a nucleic acid sequence, for example such that the original number of copies of a sequence in a sample can be determined or estimated. The redundancy (i.e., depth) of the sequencing can be determined by the length of the sequence to be determined (X), the number of sequencing reads (N), and the average read length (L). Given these parameters, the redundancy can be expressed as N×L/X. The sequencing depth/redundancy can be on the order of 2-100 units, from 100-100 units, from 1000-5000 units, or more than 5000 units, in specific examples. However, the sequencing operation(s) performed in Block S150 can additionally or alternatively be characterized by any other suitable redundancy parameter or other parameter.

In variations, sequencing in Block S150 can additionally or alternatively include methods involving targeted amplicon sequencing and/or metagenomic sequencing, implementing techniques including one or more of: sequencing-by-synthesis techniques, capillary sequencing techniques (e.g., Sanger sequencing), pyrosequencing techniques, and nanopore sequencing techniques (e.g., using an Oxford Nanopore technique). In a specific example of Blocks S140 and S150, amplification and sequencing of nucleic acids from biological samples of the set of biological samples includes: solid-phase PCR involving bridge amplification of DNA fragments of the biological samples on a substrate with oligo adapters, wherein amplification involves primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, a sequence for targeting a specific target region, a reverse index sequence (e.g., corresponding to an Illumina reverse index for MiSeq/NextSeq/HiSeq platforms), and a reverse barcode sequence. In the specific example, sequencing comprises Illumina sequencing (e.g., with a HiSeq platform, with a MiSeq platform, with a NextSeq platform, etc.) using a sequencing-by-synthesis technique.

Similar to previously described blocks, Block S150 can be implemented using a system comprising apparatus, control subsystems, and actuators that enable reception of samples, wet lab processing of samples, data extraction from samples, and/or data processing for samples with reduced human input (e.g., in an automated manner), in relation to sequencing. In some variations, such systems can include one or more of: sample container handling robotics (e.g., gantries, robotic arm systems having desired degrees of freedom and dexterity, control system), fluid delivery apparatus (e.g., aspiration systems, fluid conduits, control systems), thermocycling apparatus (e.g., heaters and heater control modules), sample analysis apparatus (e.g., optical detection systems, signal transmission units), computing systems (e.g., data storage units, remote servers for data processing, benchtop data processing computers, cloud-based computing systems, etc.), and any other suitable apparatus for semi-automated or automated sample handling. However, Block S150 can additionally or alternatively be implemented entirely by a human or other entity.

Some variations of sample processing in relation to the described blocks of the method 100 can additionally or alternatively include further purification of amplified nucleic acids (e.g., PCR products) prior to sequencing, which functions to remove excess amplification elements (e.g., primers, dNTPs, enzymes, salts, etc.). In examples, additional purification can be facilitated using any one or more of: purification kits, buffers, alcohols, pH indicators, chaotropic salts, nucleic acid binding filters, centrifugation, and any other suitable purification technique.

### 1.6 Method—Analysis

Block S160 recites: generating an analysis of the sample from the sequences determined from at least one of the first and the second subsets of oligonucleotide-conjugated antibodies, wherein the analysis is informative of a relative distribution of binding between antibodies of the set of oligonucleotide-conjugated antibodies and the set of targets. Block S160 functions to characterize which antibody-oligonucleotide complexes bind or fail to bind to target antigens of the immunoassay of Block S130, and allows identification of specific antibody-bound molecules based upon a sequencing process. As such, the analysis can enable detection of which antibody-oligonucleotide complexes bind (or fail to bind) to target antigens, and characterize relative amounts of binding between different antibodies of the set of antibody-oligonucleotide complexes, and the antigen(s)/target(s) used in Block S130. With normalization by a known factor (e.g., a normalization quantity), the analysis of Block S160 can additionally or alternatively provide absolute measures (e.g., absolute quantitative) of binding between different antibodies of the set of antibody-oligonucleotide complexes.

In an example, with a set of different antibodies used in Block S110, the analysis can output information pertaining to which of the different antibodies bind to the antigen(s)/target(s) in Block S130, and the relative or absolute amounts of each of the different antibodies that bound to target(s)/antigen(s) in Block S130, based upon the sequencing operation(s) and outputs of Block S150. As such, in combination with knowledge (e.g., antigen specificity information) pertaining to the antibodies used in Block S110, Block S160 can enable characterization of the amount and presence of different antigen(s)/target(s) within a test sample from a subject. Some variations of Block S160 can thus include receiving a supplementary dataset pertaining to antigen specificity across the set of antibodies, and generating a characterization that is indicative of a set of targets within a test sample from a subject, upon processing the supplementary dataset with the sequences provided from Block S150.

In variations, the analysis can further provide information derived from qualitative and/or quantitative measures of antibody-antigen binding behavior, as determined upon sequencing of the oligonucleotides in Block S150.

In enabling processing and analysis of which of a set of diverse antibodies bind to the target(s)/antigen(s) of the immunoassay of Block S130, Block S160 can provide a rapid and novel diagnostic tool for detection of multiple antigen targets in parallel, in a multiplex manner, and in an efficient manner. As such, as indicated above, the analysis of Block S160 can output diagnoses associated with infectious agents (e.g., prions), microorganisms (e.g., bacteria, viruses, fungal organisms, etc.), toxic compounds, and any other suitable antibody target associated with medical (e.g., diagnostic, therapeutic) or research applications. In examples, the analysis of Block S160 can support diagnostic tests for disease panels (e.g., respiratory disease panels, sexually-transmitted disease panels, etc.) and any other suitable disease panel.

In some variations, the method 100 can thus include Block S170, as shown in FIG. 5, which recites: providing a characterization of the set of targets to an entity associated with the sample, in relation to providing a diagnostic test for one or more health-related conditions of the subject. The characterization can be provided in an electronic format and/or in a non-electronic format to the entity. Furthermore, in variations, the entity can be a human entity (e.g., health care provider, physician, nurse, lab technician, relative, etc.) or a non-human entity (e.g., electronic healthcare platform/system associated with care and health records) associated with a subject. In variations, providing the characterization to the entity can include transmitting information in a manner that enables the information be accessible at an electronic device (e.g., personal computer, smart phone, head-mounted wearable computing device, wrist-mounted wearable computing device, tablet, laptop, netbook, etc.) of the entity. Additionally or alternatively, information can be provided to the individual in the form of a printed report, an electronic document (e.g., a PDF), as raw data, and/or in any other suitable form.

In a specific example, upon generation of the analysis and associated characterizations in Block S160, Block S170 can include establishing communication with an electronic device of the entity and transmitting information associated with the characterization in near-real time. In examples, communication rules associated with secure information transfer can include transmitting data over specific communication links (e.g., wireless links, wired links, etc.) under certain conditions, format of responses to requests, when to process requests, and any other suitable communication rules. Permission rules can include permission levels for third party accounts (e.g., varying access levels to diagnostic test results), user accounts (e.g., varying access levels for users to see a third party's analyses of diagnostic tests results in relation to treatment of a subject, etc.). Thus, in transmitting the information, a communication module (e.g., a hardware communication module associated with the electronic device of the entity) can receive data by way of a wired and/or wireless data link (e.g., a communicable link over Bluetooth, a communicable link over Bluetooth LTE, etc.), in response to near-real time analyses performed according to the method 100. However, any suitable type of rule controlling any suitable aspect of the method 100 and/or system 200 can be determined.

In specific examples, diagnostic tests associated with Block S170 can include tests for different identified health conditions and/or disease panels, wherein the identified health conditions and/or disease panels can be associated with one or more of: a neurological health condition, an autoimmune condition, an endocrine system condition, a mental health condition, a locomotor system condition, a metabolic (associated) disease condition, a cardiovascular disease condition, a cutaneous condition, a sexually transmitted disease, a dental health condition, a gastrointestinal health condition, and/or any other suitable condition, embodiments, variations, and examples of which are described in U.S. application Ser. No. 14/919,614 filed on 21 Oct. 2015, U.S. application Ser. No. 15/097,862 filed on 13 Apr. 2016, U.S. application Ser. No. 15/098,027 filed on 13 Apr. 2016, U.S. application Ser. No. 15/098,248 filed on 13 Apr. 2016, U.S. application Ser. No. 15/098,236 filed on 13 Apr. 2016, U.S. application Ser. No. 15/098,222 filed on 13 Apr. 2016, U.S. application Ser. No. 15/098,204 filed on 13 Apr. 2016, U.S. application Ser. No. 15/098,174 filed on 13 Apr. 2016, U.S. application Ser. No. 15/098,110 filed on 13 Apr. 2016, U.S. application Ser. No. 15/098,081 filed on 13 Apr. 2016, and U.S. application Ser. No. 15/098,153 filed on 13 Apr. 2016, which are herein incorporated in their entireties by this reference. In these specific examples, Block S170 can include providing qualitative information (e.g., positive test results, negative test results), quantitative information (e.g., quantitative parameter values associated with different detected or non-detected targets based on binding behavior), information associated with confidence in different sub-results of the diagnostic test (e.g., confidence ranges, indications of potential false positive results, indications of potential false negative results), information associated with non-conclusive results, and/or any suitable information related to each condition of the disease panel. Additionally or alternatively, Block S170 can provide information with health states not associated with diseases.

Block S170 can include steps that facilitate guiding of treatment or other responses to diagnostic testing information. For instance, in one variation, Block S170 can include automatically generating a treatment regimen in order to maintain or improve health states of a subject (e.g., in relation to positive test results). Generating of the treatment regimen can include generating the treatment regimen based upon a therapy model (e.g., as described in U.S. application Ser. No. 15/097,862 and filed on 13 Apr. 2016, which is herein incorporated in its entirety by this reference). Similar to above described blocks of the method 100, information associated with the treatment regimen can be provided to the subject or entity associated with the subject over a wired or wireless communicable link in an electronic format (e.g., within a mobile application, within a web application accessible by the entity or subject in a secure manner, etc.).

Aspects of the treatment regimen can include one or more of: microbiome modifying therapies (e.g., probiotic-based therapies, prebiotic-based therapies, phage-based therapies, small molecule-based therapies, etc.) that can shift a subject's microbiome composition and/or functional features toward a desired equilibrium state in promotion of the subject's health. In examples, treatments and/or therapies can be selected from therapies including one or more of: probiotic therapies, phage-based therapies, prebiotic therapies, small molecule-based therapies, cognitive/behavioral therapies, physical rehabilitation therapies, clinical therapies, medication-based therapies, diet-related therapies, and/or any other suitable therapies.

Therapy provision can be performed in an automated manner according to Block S170. For instance, in one specific application, outputs of Block S160 and/or S170 can be used to govern medication provision for a subject, using an automated medication dispenser of the subject. In this specific application, information derived from the therapy regimen can be transformed into rules for governing dispensing functions of the medication dispenser (e.g., in relation to medication dosages, in relation to medication interactions, in relation to improving/worsening health conditions states, in relation to medication titration, etc.). Identification of positive test results, for instance, can produce rules that are transmitted (e.g., over a wireless communication link, over a wired communication link) to a connected medication dispenser, such that the rules enable automated dispensing of one or more new medications to the subject with desired dosing requirements, based on the positive test results. Identification of negative test results for instance, can produce rules that are transmitted (e.g., over a wireless communication link, over a wired communication link) to a connected medication dispenser, such that the rules stop automated dispensing of one or more medications to the subject, based on the negative test results. Identification of positive test results, for instance, can additionally or alternatively produce rules that are transmitted (e.g., over a wireless communication link, over a wired communication link) to a connected medication dispenser, such that the rules enable automated dispensing of an alternative medication that has less adverse medication interaction characteristics with medications currently used by the subject, based on the positive test results.

The method 100 can, however, include any other suitable blocks or steps configured to facilitate parallel/multiplex processing of oligonucleotide-conjugated antibody samples, and analyzing data derived from antibody binding in relation to interactions between the oligonucleotide-conjugated antibodies and one or more target(s)/antigen(s). The method 100 can additionally or alternatively support other methods for detection of antibody binding to targets/antigens, and/or any other suitable methods involving sequencing of nucleic acids.

### 2. System

As shown in FIGS. 2 and 6, a system 200 for characterization of antibody binding behavior can include: a sample handling network 210 that facilitates reception of a sample from a subject, processing of the sample (e.g., in relation to oligonucleotide conjugation and antibody-target binding) at a sample processing subsystem 230 within the sample handling network, performs sequencing operations at a processing system 240 within the sample handling network, and transmits information (e.g., binding characterizations, diagnostic test information) derived from the sequencing operations to associated entities over communicable links (e.g., secure wireless communication links, secure wired communication links). The method 100 can, however, alternatively be implemented using any other suitable system(s) configured to receive and process samples, in aggregation with other information, in order to generate and share insights derived from characterizing antibody binding behavior, in a multiplexed manner.

The sample handling network 210 can functions as a platform from which sampling kits can be distributed in order to receive samples from subjects, wherein the samples can be returned for processing and analysis. One aspect of the sample handling network 210 thus functions as a distribution and receiving hub for sample handling, wherein individuals are able to transmit samples directly to the sample handling network without requiring direct contact between individuals and a clinical or laboratory-based intermediary staffed with trained personnel for biological sample handling. The sample handling network 210 is thus preferably configured to provide instructions directly to individuals pertaining to sample provision in a dependable manner without involving laboratory-trained personnel in the sample provision process, and is preferably configured to associate samples with individuals providing the samples in a secure and reliable manner that is compliant with regulatory standards (e.g., compliant with the Health Insurance Portability and Accountability Act, HIPAA). However, the sample handling network 210 can alternatively be configured to distribute sampling kits and/or receive samples from individuals using a laboratory-based or clinical-based intermediary, and/or handle samples in any other suitable manner.

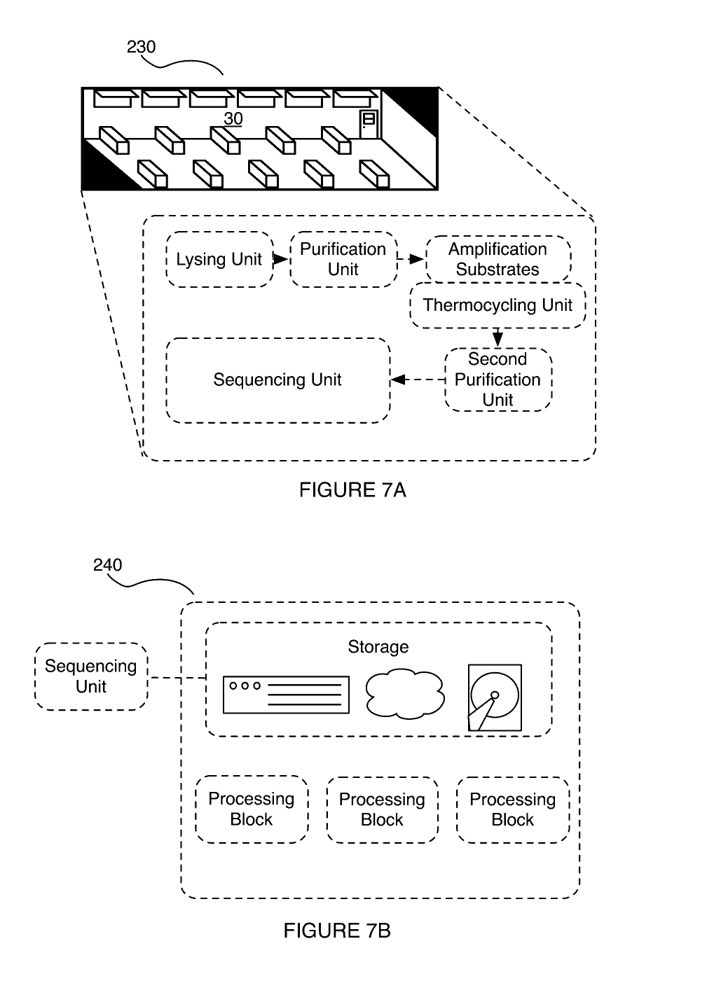

The sample processing subsystem 230, an example of which is shown in FIG. 7A, is preferably configured to process samples within the sample handling network 210, but can additionally or alternatively be configured to process samples in any other suitable network associated with the sample handling network 210. The sample processing subsystem 230 can comprise a laboratory environment 30 (e.g., wet laboratory environment) within sample handling network 210, wherein samples in sample containers received at the sample handling network 210 are transmitted within the sample handling network 210 to the sample processing subsystem 230 for sample processing (e.g., purification of nucleic acid content, amplification of nucleic acid content, sequencing of nucleic acid content). The sample processing subsystem 230 is preferably implemented entirely within the sample handling network 210, but can additionally or alternatively include sub-modules that are implemented within the sample handling network 210 (e.g., in an “in house” manner) and sub-modules that are implemented outside of the sample handling network 210 (e.g., in an “out of house” manner). In one variation, sample purification can be performed at a first sub-module of the sample processing subsystem 230 within the sample handling network 210, amplification can be performed at a second sub-module of the sample processing subsystem 230 outside of the sample handling network 210, and sequencing can be performed at a third sub-module of the sample processing subsystem 230 outside of the sample handling network 210. The sample processing subsystem 230 and sub-modules thereof can, however, be configured in any other suitable manner in relation to the sample handling network 210.

For sample processing and purification, the sample processing subsystem 230 preferably comprises an environment 30 (e.g., sterilized laboratory hood, sterilized room) sterilized of any contaminating substances (e.g., substances that could affect nucleic acids in a sample or contribute to contaminant nucleic acids), wherein sample processing is conducted. The environment 30 can be temperature controlled, controlled for oxygen content, controlled for carbon dioxide content, and/or controlled for light exposure (e.g., exposure to ultraviolet light). A purification module 232 of the sample processing subsystem 230 can operate based upon force-based separation, sized-based separation, binding-moiety-based separation (e.g., with magnetic binding moieties, with buoyant binding moieties, etc.), and/or any other suitable form of separation. For instance, a purification module 232 can include one or more of: a centrifuge to facilitate extraction of a supernatant, a filter (e.g., a filtration plate), a fluid delivery module configured to combine a lysed sample with moieties that bind to targets and/or waste material of a sample, a wash reagent delivery system, an elution reagent delivery system, and any other suitable apparatus for purification of target content from a sample.

For nucleic acid amplification (e.g., associated with oligonucleotide amplification), the sample processing subsystem 230 can comprise amplification substrates 233 (e.g., PCR-compatible sample-receiving substrates) and a thermocycling module 234 configured to perform thermocycling on the amplification substrates 233, wherein the amplification substrates 233 are configured to receive one or more samples (e.g., lysed samples), primer solutions, reagents (e.g., a master mix, PCR water), and any other suitable materials for nucleic acid amplification. The thermocycling module 234 can be configured to thermocycle different amplification substrates according to individualized thermocycling sequences (e.g., temperatures, ramp up times, hold times, ramp down times, cycles, etc.) using an array of individually controllable heating elements, or can additionally or alternatively be configured to thermocycle different amplification substrates according to common thermocycling sequences using a single heating element or an array of co-controlled heating elements. The sample processing subsystem 230 can additionally or alternatively include a second purification module 235 configured to purify nucleic acid amplification products from amplification reagents (e.g., excess primers, excess dNTPs, enzymes, salts, etc.). In variations, the purification module 235 can include purification kits comprising buffers, alcohols (e.g., ethanol, isopropanol, etc.), pH indicators, chaotropic salts, nucleic acid binding filters, and centrifugation. The sample processing subsystem 230 can, however, comprise any other suitable elements (e.g., spectrophotometric apparatus for quantitation, fluorescence modules for quantitation using fluorescent dyes that bind to nucleic acids, capillary elements for size selection, electrophoretic elements for size selection, filtration elements for size selection, quality control elements, etc.).

For sequencing of amplified nucleic acids, the sample processing subsystem 230 can comprise a sequencing module 236 that operates according to one of: sequencing-by-synthesis techniques (e.g., Illumina sequencing), capillary sequencing techniques (e.g., Sanger sequencing), pyrosequencing techniques, single-molecule real-time (SMRT) techniques, sequencing by ligation (e.g., SOLiD) techniques, reversible terminator sequencing techniques, proton detection sequencing techniques, ion semiconductor (e.g., Ion Torrent) sequencing techniques, nanopore sequencing techniques, electronic sequencing techniques, and any other suitable type of sequencing technique. In specific examples, the sequencing module 236 of the sample processing subsystem 230 can include one or more of: an Applied Biosystems® ABI 3730 DNA Analyzer, a 454 Life Sciences® 454 FLX Titanium sequencer, an Illumina® sequencer (e.g., a GAIIx sequencer, a HiSeq sequencer, a MiSeq sequencer), a Pacific Biosciences® PacBio sequencer, an Ion Torrent™ sequencer, and any other suitable sequencer.

Elements of the sample processing subsystem 230 can be configured to operate in an automated manner, and in one example, the sample processing subsystem 230 comprises a laboratory automation workstation (e.g., a Biomek® Laboratory Automation Workstation) which automates sample container handling and processing by way of robotic arms and gantries, actuators, and fluid delivery systems governed by one or more control modules. Alternatively the sample processing subsystem 230 can be configured to be operated at least in part by a trained technician, in order to provide manual or semi-manual forms of sample handling and processing. Furthermore, the sample processing subsystem 230 can be configured to operate in a continuous-flow manner by using fluidic devices (e.g., microfluidic devices) that enable multiple blocks of processing (e.g., sample lysing, nucleic acid extraction, nucleic acid purification, nucleic acid amplification, etc.) to be performed on a single fluidic device. Alternatively, elements of the sample processing subsystem 230 can be configured to operate more discretely using different devices and/or different sample process chambers.

The processing system 240, an example of which is shown in FIG. 7B, is configured to perform analyses according to blocks of the method 100 described in Section 1 above. The processing system 240 can be in direct communication with modules of the sample processing subsystem 230, and in one variation, a sequencing module 236 of the sample handling network 210 can be configured to provide sequenced data as an output to a module of the processing system 240. Additionally or alternatively, the processing system 240 can be configured to receive inputs from outputs of the sample processing subsystem 230 by way of a storage device 241 configured to store data derived from processing of samples received at the sample handling network 210. The processing system 240 is preferably implemented in one or more computing systems, wherein the computing system(s) can be implemented at least in part in the cloud and/or as a machine (e.g., computing machine, server, cloud-based computing system etc.) configured to receive a computer-readable medium storing computer-readable instructions. As such, the processing system 240 can comprise one or more processing modules, implemented in the cloud and/or as machine, comprising instructions for performing blocks of the method 100. The processing system 240 can, however, be configured in any other suitable manner.

The method 100 and/or system of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the embodiments of the invention without departing from the scope of this invention as defined in the following claims.

We claim:

1. A method for characterization of antibody-bound targets by sequencing of synthetic oligonucleotides, comprising:
conjugating each of a set of antibodies with a synthetic oligonucleotide, thereby generating a set of oligonucleotide-conjugated antibodies;
binding at least a first subset of the set of oligonucleotide-conjugated antibodies to a set of targets at a capture substrate, the set of targets associated with a sample from a subject;
determining a sequence for at least one of: 1) each oligonucleotide of the first subset of oligonucleotide-conjugated antibodies that bind to the set of targets and 2) each oligonucleotide of a second subset of the set of oligonucleotide-conjugated antibodies that fail to bind to the set of targets; and
generating an analysis of the sample from the sequences determined from at least one of the first and the second subsets of oligonucleotide-conjugated antibodies, wherein the analysis identifies the set of targets based upon sequencing of each oligonucleotide of at least on of the first and the second subsets, and wherein the analysis is informative of a relative distribution of binding between antibodies of the set of oligonucleotide-conjugated antibodies and the set of targets.

2. The method of claim 1, wherein the set of antibodies includes a diverse group of different types of antibodies associated with a diverse set of targets in an identified health condition.

3. The method of claim 2, wherein the identified health condition includes at least one of: a neurological health condition, an autoimmune condition, an endocrine system condition, a mental health condition, a locomotor system condition, a metabolic-associated disease condition, a cardiovascular disease condition, a cutaneous condition, a sexually transmitted disease, a dental health condition, and a gastrointestinal health condition.

4. The method of claim 1, wherein conjugating each of the set of antibodies with the synthetic oligonucleotide includes conjugating each of the set of antibodies with a synthetic oligonucleotide having at least a 16S V4-like region, and wherein the method further includes simultaneously amplifying all bacterial and archaeal DNA present in a sample, in addition to the synthetic oligonucleotides, with 16S-compatible primers, prior to sequencing.

5. The method of claim 1, further including: pre-processing the set of antibodies with solvents that reduce interactions between different antibodies of the set of antibodies prior to oligonucleotide conjugation.

6. The method of claim 1, further including: pre-processing the set of antibodies with blocking compounds that controllably block antibody sites having potential for interacting with other antibodies of the set of antibodies prior to oligonucleotide conjugation.

7. The method of claim 1, further including pre-processing the oligonucleotides associated with the set of antibodies with: elimination of compounds having functional groups that compete for conjugation sites, minimization of base repetition in the oligonucleotides, and implementation of oligonucleotides with less than or equal to four sequential Guanine bases.

8. The method of claim 1, further including processing the oligonucleotides associated with the set of antibodies with a terminal amino group prior to conjugation with the set of antibodies, and removing unbound oligonucleotide components post-conjugation with the set of antibodies.

9. The method of claim 1, wherein binding a first subset of the set of oligonucleotide-conjugated antibodies to a set of targets at a capture substrate comprises binding the first subset in an enzyme-linked immunosorbent assay (ELISA) configuration.

10. The method of claim 1, wherein each of the set of oligonucleotide-conjugated antibodies bound to targets is analyzed with a sequencing operation, with or without amplification.

11. The method of claim 1, further including: from the analysis, generating a diagnostic characterization indicative of positive identification of at least one health condition associated with the sample from the subject.

12. The method of claim 11, wherein generating the analysis comprises determining which of the set of antibody-oligonucleotide complexes bind to targets of the set of targets and determining relative amounts of binding between different antibodies of the set of antibody-oligonucleotide complexes to the set of targets, and wherein generating the diagnostic characterization comprises contemporaneously generating diagnostics associated with a panel of conditions tested in the subject.

13. The method of claim 11, further including: based upon the diagnostic characterization, automatically promoting a treatment regimen to the subject with at least one health condition.

14. The method of claim 13, wherein promoting the treatment regimen comprises promoting a therapy to the subject, wherein promoting the therapy comprises promoting a microbiome modifying therapy to a subject in order to treat at least one condition of the subject.

15. The method of claim 14, wherein promoting the microbiome modifying therapy comprises promoting at least one of a prebiotic therapy and a probiotic therapy to the subject, wherein the microbiome modifying therapy comprises a consumable that selectively modulates a population size of a desired taxon or the abundance of a desired function for treatment of at least one condition of the subject.

16. A method for characterization of antibody-bound targets by sequencing of synthetic oligonucleotides, the method comprising:
conjugating each of a set of antibodies with a synthetic oligonucleotide having at least one of: a 16S-like region and another characteristic gene region, thereby generating a set of oligonucleotide-conjugated antibodies;
contemporaneously binding at least a first subset of the set of oligonucleotide-conjugated antibodies to a set of targets at a capture substrate, the set of targets associated with a sample from a subject;
contemporaneously determining a sequence for each oligonucleotide of at least one of the first subset of oligonucleotide-conjugated antibodies that bind to the set of targets and a second subset of oligonucleotide-conjugated antibodies that fail to bind to the set of targets;
generating an analysis of the sample from the sequences determined from at least one of the first and the second subsets of oligonucleotide-conjugated antibodies, wherein the analysis includes an identification of targets bound to the first subset of oligonucleotide-conjugated antibodies upon sequencing of oligonucleotides, and wherein the analysis is informative of a relative distribution of binding between antibodies of the set of oligonucleotide-conjugated antibodies and the set of targets;
from the analysis, generating a diagnostic characterization indicative of positive identification of at least one health condition associated with the sample from the subject;
automatically transmitting the diagnostic characterization to at least an entity associated with the subject, upon establishing a communicable link with an electronic device of the entity; and
based upon the diagnostic characterization, automatically promoting a treatment regimen to the subject with at least one health condition.

17. The method of claim 16, wherein the set of antibodies includes a diverse group of different types of antibodies associated with a diverse set of targets in at least one identified health condition

18. The method of claim 17, wherein the identified health condition includes at least one of: a neurological health condition, an autoimmune condition, an endocrine system condition, a mental health condition, a locomotor system condition, a metabolic-associated disease condition, a cardiovascular disease condition, a cutaneous condition, a sexually transmitted disease, a dental health condition, and a gastrointestinal health condition.

19. The method of claim 17, further including pre-processing the set of antibodies with at least one of: blocking compounds that controllably block antibody sites having potential for interacting with other antibodies of the set of antibodies, solvents that reduce interactions between different antibodies of the set of antibodies, and selective introduction of antibodies that reduce interactions between different antibodies of the set of antibodies.

20. The method of claim 17, further including processing the oligonucleotides associated with the set of antibodies with a terminal amino group prior to conjugation with the set of antibodies, and removing unbound oligonucleotide components post-conjugation with the set of antibodies.

21. The method of claim 17, wherein binding a first subset of the set of oligonucleotide-conjugated antibodies to a set of targets at a capture substrate comprises binding the first subset in an enzyme-linked immunosorbent assay (ELISA) configuration.

22. The method of claim 17, wherein generating the analysis comprises determining which of the set of antibody-oligonucleotide complexes bind to targets of the set of targets and determining relative amounts of binding between different antibodies of the set of antibody-oligonucleotide complexes to the set of targets, and wherein generating the diagnostic characterization comprises contemporaneously generating diagnostics associated with a panel of conditions tested in the subject.

23. The method of claim 16, wherein promoting the treatment regimen comprises promoting a therapy to the subject, wherein promoting the therapy comprises promoting a microbiome modifying therapy to the subject for the treatment of the at least one condition of the subject.

24. The method of claim 16, wherein conjugating each of the set of antibodies with the synthetic oligonucleotide comprises conjugating in at least one of a reversible manner and an irreversible manner.

25. The method of claim 16, wherein conjugating comprises at least one of: mixing the set of antibodies with oligonucleotides in solution, incubating the set of antibodies with oligonucleotides in solution, and performing unidirectional conjugation in order to form antibody-oligonucleotide complexes without formation of antibody-antibody complexes and without formation of oligonucleotide-oligonucleotide complexes.

26. The method of claim 16, wherein each of the set of oligonucleotide-conjugated antibodies bound to targets is analyzed with a sequencing operation, with or without amplification.

27. The method of claim 16, wherein determining a sequence for each oligonucleotide comprises using a PCR-based process.
